(19) United States
(12) Patent Application Publication
(10) Pub. No.: US 2006/0105441 A1
(43) Pub. Date: May 18, 2006

(54) Process for the preparation of indole derivatives by enzymatic acylation

(76) Inventors: Reinhold Ohrlein, Rheinfelden-Herten (DE); Nicole End, Oberwil (CH); Gabriele Baisch, Binzen (DE)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/548,356

(22) PCT Filed: Mar. 3, 2004
(86) PCT No.: PCT/EP04/50244

§ 371 Date: Sep. 7, 2005

(30) Foreign Application Priority Data
Mar. 13, 2003 (EP) 03405174.8

(51) Int. Cl. C12P 7/40; C07D 209/18

(52) U.S. Cl. 435/136; 548/494

(57) ABSTRACT

A process for the preparation of compounds of formula (1), by enzymatic acylation to form compounds of formulae (3a) and (3a) and then reacting the compound of formula (3a) with a compound introducing the radical of formula —CH₂—COOR₈, R₈ being an organic radical, and then reducing, and optionally hydrolysing, the resulting compound of formula (4).

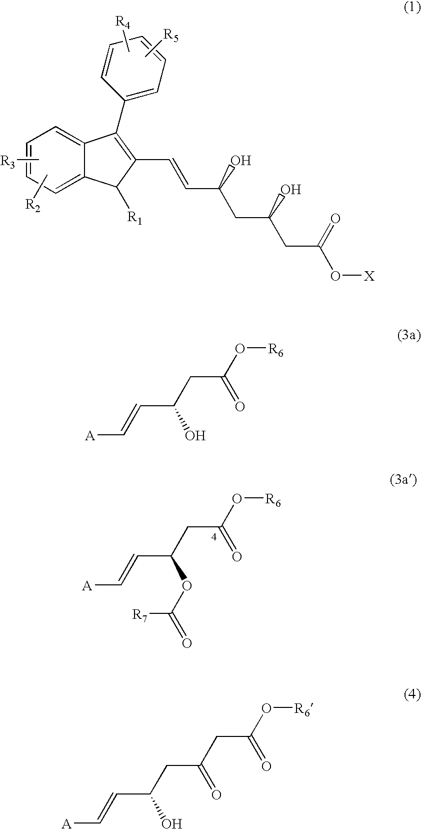

The present invention relates to a process for the preparation of indole derivatives and to novel intermediates.

Indole derivatives of formula (1) hereinbelow are known as pharmaceutical active ingredients or as precursors for the preparation thereof, for example from U.S. Pat. No. 4,739,073 or WO 01/92223. An important indole derivative is fluvastatin, an HMG-CoA reductase inhibitor, that is to say an inhibitor of cholesterol biosynthesis, which is used in the treatment of hyperlipoproteinaemia and arteriosclerosis.

Known processes for the preparation of optically active indole compounds of formula (1) do not in all cases meet the demands that are made of the yield and economic viability of the processes.

The present Application is consequently based on the problem of making available a novel process for the preparation of indole compounds of formula (1), by means of which such compounds can be obtained in as high a yield as possible and with good economic viability.

The present invention accordingly relates to a process for the preparation of compounds of formula (1)

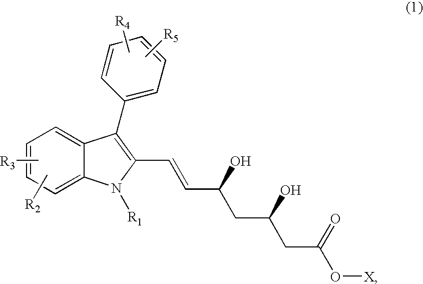

wherein R₁ is unsubstituted or substituted C₁-C₈alkyl,

R₂, R₃, R₄ and R₅ are each independently of the others hydrogen, unsubstituted or substituted C₁-C₈alkyl, C₁-C₈alkoxy, phenoxy or benzyloxy, or halogen, and

X is hydrogen, an organic radical or a cation,

which process comprises reacting a compound of formula (2)

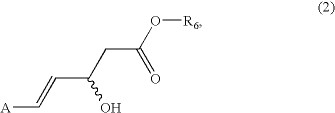

wherein R₆ is an organic radical and

A is an organic radical of formula (1a)

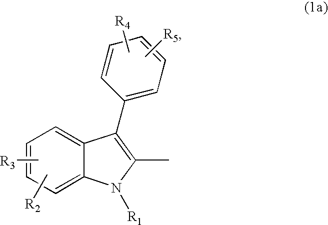

wherein R₁, R₂, R₃, R₄ and R₅ are as defined hereinbefore,

by enzymatic acylation to form compounds of formulae (3a) and (3a′)

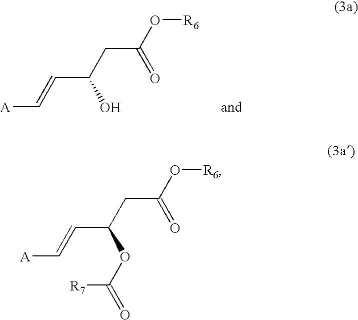

wherein

A is an organic radical of formula (1a) and

R₆ and R₇ are each independently of the other an organic radical, and

then reacting the compound of formula (3a) with a compound introducing the radical of formula —CH₂—COOR₈, R₈ being an organic radical, and

then reducing, and optionally hydrolysing, the resulting compound of formula (4)

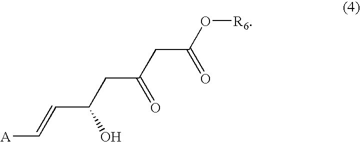

As C₁-C₈alkyl radicals for R₁ there come into consideration, for example, methyl, ethyl, n- or iso-propyl, n-, sec- or tert-butyl, or straight-chain or branched pentyl, hexyl, heptyl or octyl. C₁-C₄Alkyl radicals are preferred. R₁ is preferably propyl, especially isopropyl.

As C₁-C₈alkyl radicals for R₂, R₃, R₄ and R₅ there come into consideration, for example, methyl, ethyl, n- or iso-propyl, n-, sec- or tert-butyl, or straight-chain or branched pentyl, hexyl, heptyl or octyl. The mentioned alkyl radicals may be unsubstituted or substituted by, for example, halogen, e.g. fluorine. Corresponding C₁-C₄alkyl radicals are preferred.

As C₁-C₈alkyl radicals for X, R₆, R₇ and R₈ there come into consideration, for example, methyl, ethyl, n- or iso-propyl, n-, sec- or tert-butyl, or straight-chain or branched pentyl, hexyl, heptyl or octyl. As C₁-C₆alkyl radicals there come into consideration, for example, methyl, ethyl, n- or iso-propyl, n-, sec- or tert-butyl, or straight-chain or branched pentyl or hexyl. C₁-C₄Akyl radicals are, for example, methyl, ethyl, n- or iso-propyl, n-, sec- or tert-butyl.

As C₁-C₈alkoxy radicals for R₂, R₃, R₄ and R₅ there come into consideration especially C₁-C₄alkoxy radicals such as, for example, methoxy or ethoxy.

As halogen for R₂, R₃, R₄ and R₅ there come into consideration, for example, fluorine or chlorine, especially fluorine.

R₂, R₃ and R₅ are preferably hydrogen. R₄ is preferably fluorine, especially fluorine bonded in the 4-position.

As organic radicals for X, Re, R₇ and R₈, each independently of the others, there come into consideration, for example, unsubstituted or substituted alkyl, alkenyl, alkynyl or phenyl radicals. Special mention may be made of unsubstituted or substituted C₁-C₁₂alkyl, C₃-C₁₂alkenyl, C₃-C₁₂alkynyl or phenyl radicals. For X, R₆, R₇ and R₈, each independently of the others, preference is given to unsubstituted or substituted alkyl radicals, preferably C₁-C₁₂alkyl radicals, especially C₁-C₈alkyl radicals, more especially C₁-C₆alkyl radicals and very especially C₁-C₄alkyl radicals such as, for example, methyl, ethyl, n- or iso-propyl, n-, sec- or tert-butyl. As examples of substituents of the alkyl radicals there may be mentioned C₁-C₄alkyl, C₁-C₄alkoxy, nitro, halogen or hydroxy, or phenyl which may, for example, be further substituted on the phenyl ring by C₁-C₄alkyl, C₁-C₄alkoxy, nitro, halogen or by hydroxy. As examples of X, R₆, R₇ and R₈, each independently of the others, there may be mentioned methyl, ethyl, n- or iso-propyl, n-, sec- or tert-butyl, allyl, a methyl methyl ether, methyl ethyl ether or ethyl methyl ether radical, benzyl, nitrobenzyl and hydroxybenzyl. Special preference is given to X being C₁-C₄alkyl, especially butyl and preferably tert-butyl. Special preference is given to Re being a C₁-C₆alkyl radical, especially methyl, ethyl, n- or iso-propyl, n-, sec- or tert-butyl, more especially ethyl and isopropyl, very especially ethyl. Preference is furthermore given to R₇ being a C₁-C₄alkoxy-C₁-C₄alkylene or C₁-C₈alkyl radical.

Special preference is given to R₇ being a methoxymethylene radical or a C₁-C₈alkyl radical, especially methyl, ethyl, n- or iso-propyl, or n- or sec-butyl, more especially methyl, ethyl and n-propyl.

Special preference is given to R₈ being a C₁-C₈alkyl radical, especially methyl, ethyl, n- or iso-propyl, or n-, sec-butyl or tert-butyl, more especially ethyl, isopropyl and tert-butyl.

When the radical X is a cation, the cation may be, for example, sodium or potassium, especially sodium.

Preference is given to X being hydrogen, C₁-C₈alkyl which is unsubstituted or substituted by phenyl, or a cation. Very special preference is given to X being a cation, such as sodium or potassium, especially sodium.

Enzymes that are used in the enzymatic acylation are, for example, esterases, lipases or proteases. Those enzymes are known and are described, for example, by U. T. Bornscheuer, R. T. Kazlauskas in “Hydrolases in Organic Synthesis” appearing in Wiley-VCH Verlag, 1999, pages 65-195, ISBN 3-527-30104-6.

Esterases and lipases may originate, for example, from animals, micro-organisms or fungi. Esterases from animals are, for example, pig liver esterase, PLE, and pig pancreas esterase, PPE.

Esterases from micro-organisms or fungi are, for example, Bacillus subtilis esterase, Pichia esterases, yeast esterases, Rhizopus species esterases, and Penicillium species esterases. Lipases from animals are, for example, pig pancreas lipase, PPL.

Lipases from fungi and micro-organisms are, for example, G. candidum, GCL, H. lanuginosa, HLL, Rhizopus species, RML or ROL, Candida species, CAL-A, CAL-B or CCL, Aspergillus species, ANL, and Pseudomonas species, PCL or PFL.

Proteases are, for example, amidases, and are, for example, preferably subblisin, thermitase, chymotrypsin, thermolysin, papain, aminoacylases, penicillin amidases or trypsin. The enzymes can be obtained in the form of crude isolates and/or in purified form from natural sources and/or from micro-organisms by modern cloning procedures by means of overexpression and amplification.

The enzymes may be as such or immobilised or adsorbed on a variety of supports, for example on silica gel, kieselguhr, Celite® or Eupergit®, as marketed by, for example, the Rbhm&Haas company from Darmstadt in Germany.

Cross-linked enzymes, CLEC, can also be used, as sold by, for example, the ALTUS BIOLOGICS company.

The possibilities for using immobilised enzymes are described, for example, by U. T. Bornscheuer and R. T. Kazlauskas in “Hydrolases in Organic Synthesis”, appearing in Wiley-VCH Verlag, 1999, pages 61-64, ISBN 3-527-30104-6, or by K. Faber in “Biotransformation in Organic Chemistry”, appearing in Springer Verlag, 1997, 3rd edition, pages 345-357, ISBN 3-540-61688-8; or by H.-J. Rehm and G. Reed in “Biotechnology”, appearing in VCH Verlag, 1998, 2nd Vol., pages 407-411.

Special preference is given to immobilised lipases that have a certain degree of thermostability and longevity such as, for example, Novozyme 435 [recombinant Candida antarctica lipase B, as described, for example, by E. M. Anderson et al. in “Biocatalytic Biotransformation”, 1998, 16, page 181], or the lipozyme RM-IM (R. miehei).

The enzymes QLM, QL are obtainable from the Meito Sangyo company in Japan. Preference is given to commercially available enzymes and also to those that are described, for example, in H.-J. Rehm and G. Reed in “Biotechnology”, appearing in VCH Verlag, 1998, Volume 2, pages 40-42.

Special preference is given to the lipases Novozyme 435 and Lipozyme RM-IM, which are obtainable, for example, from the NOVO Nordisk company, Bagswaerd, Denmark.

The compounds of formula (2) are known and can be prepared, for example, in analogy to the process as in, for example, WO 01/92223, Example 16, in conjunction with prior Examples in WO 01/92223 for synthesis of the starting materials in question.

The acylation of the compound of formula (2) to form compounds of formulae (3a) and (3a′) in the presence of enzymes is novel.

As acylation agent there are used the generally known compounds that are suitable for transferring an acyl group such as, for example, active esters, alkanolates, enol esters, anhydrides and non-activated esters.

Preference is given to active esters as described, for example, by K. Faber in “Biotransformations in Organic Synthesis”, Springer Verlag 1997, ISBN 3-540-61688-8, 3rd edition, especially on pages 309-328.

Especially preferred active esters are vinyl esters and isopropenyl esters of formula

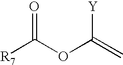

wherein

R₇ has the definitions and preferred meanings given above, and

Y is hydrogen or methyl.

The enzymes can be used with or without solvent.

As solvent there are usually used organic solvents such as, for example, hexane, toluene, benzene, tetrahydrofuran, diethyl ether, methyl tert-butyl ether or methylene chloride etc. or mixtures of those solvents.

The enzymes are used preferably without solvent or with only small amounts of solvents.

The reaction temperature is usually in the range from 283 to 353K, preferably from 298 to 343K.

The amount of the enzyme used and the concentrations of the starting materials are dependent upon the reaction conditions selected in the particular case such as, for example, temperature, reaction time and solvent.

It has proved to be advantageous to carry out the reactions under slightly reduced pressure, in a pressure range from 0.1 to 200 mbar.

If desired, the compounds of formula (3a) and/or (3a′) may be isolated and purified. Isolation and purification are carried out in accordance with generally known methods, for example by washing with an alkaline solution in the pH range from pH 8 to pH 12, preferably with an alkali metal carbonate solution or an alkali metal hydrogen carbonate solution or an alkali metal hydroxide solution, preferably a sodium hydrogen carbonate solution. Subsequent washing with an alkali metal salt solution, preferably sodium chloride solution, may be advantageous. If desired, after washing, the product is concentrated by evaporation, dried and, if necessary, chromatographically purified.

An alkali metal carbonate solution is, for example, a sodium, potassium or lithium carbonate solution.

An alkali metal hydrogen carbonate solution is, for example, a sodium, potassium or lithium hydrogen carbonate solution.

An alkali metal hydroxide solution is, for example, a sodium, potassium or lithium hydroxide solution.

An alkali metal salt solution is, for example, a sodium, potassium or lithium halide solution.

The reaction of the compound of formula (3a) to form the compound of formula (4) can be carried out, for example, in accordance with the process described in U.S. Pat. No. 4,870,199. For example, as the compound introducing the radical of formula —CH₂—COOR₈ there may be used a compound of formula CH₃—COOR₈, such as tert-butyl acetate, R₈ having the definitions and preferred meanings given above. In general, the reaction is so performed that, in the presence of a strong base, such as lithium diisopropylamide, a monoanion of the compound of formula CH₃—COOR₈ is formed. The reaction is usually carried out in an anhydrous, inert, organic solvent, for example an ether such as diethyl ether, 1,2-dimethoxy-ethane, 1,2-diethoxyethane or, especially, tetrahydrofuran, the reaction generally being carried out under an inert gas atmosphere at a temperature of from −80 to 25° C. In a next step, the monoanion formed is reacted with the compound of formula (3a), that reaction usually being carried out in the same solvent and under an inert gas atmosphere at a temperature of, for example, from −80 to 25° C.

The reduction of the compound of formula (4) can be carried out, for example, by means of a cyclic boronate using sodium borohydride, as in O. Tempkin, Tetrahedron, Vol. 53, No. 31, 10659-10670 (1997). The reduction is carried out, for example, in an ether and/or lower alcohol, such as tetrahydrofuran or methanol, at a temperature of, for example, from −50 to −80° C. As borane there comes into consideration, for example, diethyl methoxyborane. Furthermore, the reduction can also be carried out using diisobutylaluminium hydride or tributyltin hydride, as in S. Kiyooka, Tetrahedron Letters, Vol. 27, No. 26, 3009-3012 (1986), or using zinc borohydride, as described in F. Kathawala, Helv. Chim. Acta, Vol. 69, 803-805 (1986). The reduction can also be carried out using NaBH₄ in the presence of triethyl boranes as complexing agents, as described in U.S. Pat. No. 4,739,073.

When X is hydrogen or an organic radical, X can be converted into a cation, for example by hydrolysis.

The hydrolysis of the compound obtained after reduction of the compound of formula (4) can be carried out, for example, by conventional basic hydrolysis of the esters. For that purpose, the compound obtained after reduction of the compound of formula (4) is treated with approximately one mol of an inorganic base such as, for example, an alkali metal hydroxide, especially sodium hydroxide, in a mixture of water and a water-miscible organic solvent such as, for example, a lower alcohol or an ether, such as methanol, ethanol or tetrahydrofuran, at a temperature of, for example, from 0 to 80° C. It is also possible to use a slight excess of base and then to remove the excess of ester by extracting with a water-immiscible organic solvent, for example tert-butyl methyl ether; freeze-drying may be carried out subsequently. For the purpose of formation of the free acid, the ester can also be hydrolysed in an acidic medium, it being possible to carry out such hydrolysis in accordance with processes known per se. Preference is given to carrying out hydrolysis, preferably using sodium hydroxide, after the reduction of the compound of formula (4).

An alkali metal hydroxide is, for example, a sodium, potassium or lithium hydroxide.

The invention relates also to a process for the preparation of compounds of formulae (3a) and (3a′) by reacting a compound of formula (2) by means of enzymatic acylation to form compounds of formulae (3a) and (3a′).

Preference is given to the enzymatic acylations according to the invention wherein a compound of formula (2) is reacted by enzymatic acylation in the presence of immobilised or insoluble enzymes to form compounds of formulae (3a) and (3a′).

Special preference is given to the enzymatic acylations according to the invention, especially using active esters, wherein a racemic compound of formula (2) is reacted by enzymatic acylation in the presence of immobilised or insoluble enzymes in a continuous process to form compounds of formulae (3a) and (3a′).

The use of immobilised or insoluble enzymes in a continuous process is described, for example, in W. Tischer et al. in “TIBTECH”, 1999, 17, 326; or in J. Lalonde, Curr. Opin. in “Drug Disc. & Development” 1998, 1(3), 271.

Continuous processes are performed in suitable reactors as described, for example, by V. M. Balcao et al. in “Enzyme Microbiological Technology”, 1996, 18 392; or by L. Giorno et al. in “TIBTECH”, 2000, 18, 339.

In continuous processes the enzymes can be used over several reaction cycles and can be readily separated off from the desired products.

The compounds of formula (3a) wherein R₆ is ethyl and also compounds of formula (3a′) are novel.

The present invention relates also to compounds of formula (3a) wherein R₈ is ethyl and also to compounds of formula (3a′).

The compounds of formulae (3a) and (3a′) are also obtainable from their racemates. The racemate may be resolved, for example by means of known processes for enantiomer separation, into the optically pure antipodes, for example by means of preparative chromatography on chiral supports (HPLC) or by esterifying and crystallising out using optically pure precipitating agents.

The optical purity of the compound of formula (3a) used influences the optical purity of the compounds of formula (1). The optical purity of the compounds of formulae (3a) and (3a′) and of the compound of formula (1) is at least 60%, especially 80% and preferably 90%. Special preference is given to the optical purity of the compounds of formulae (3a), (3a′) and (1) being at least 95%, preferably 97.5% and especially 99%.

The racemate of compounds of formula (3a′) is obtainable according to generally known methods from a compound of formula (2) in the presence of an acylating agent and a promoter. The promoter is, for example, pyridine or triethylamine (see U.S. Pat. No. 4,739,073, column 10, compound of formula (XI) and column 21, lines 45 to 57), or an acid such as, for example, boron trifluoride or aluminium trichloride; or N,N′-carbonyldiimidazole, dicyclohexylcarbodiimide, trifluoroacetic anhydride or scandium(III) trifluoromethane-sulfonate, and preferably scandium(III) trifluoromethanesulfonate.

The present invention relates also to the use of the compounds of formula (3a) or (3a′) as intermediates for the preparation of the compound of formula (1).

The following Examples illustrate the invention:

### EXAMPLE 1

### Preparation of the Racemic Compound of Formula (2)

### 5-[3-(4-Fluoro-phenyl)-1-isopropyl-1H-indol-2-yl]-3-hydroxy-pent-4-enoic acid ethyl ester

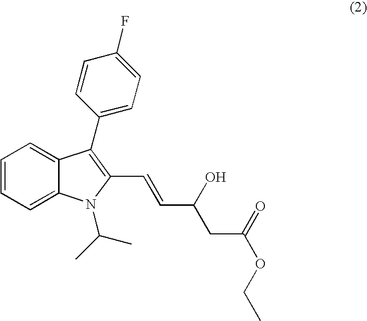

42 ml (0.3 mol) of diisopropylamine are added dropwise to a solution of 188 ml of butyllithium (1.6M in hexane) (0.3 mol) in 250 ml of tetrahydrofuran at −60° C. over a period of 15 minutes. Then a solution of 29 ml (0.3 mol) of ethyl acetate in 220 ml of tetrahydrofuran is added, also at −60° C., over a period of 1 hour. The reaction mixture is stirred for a further 30 minutes and then, within a period of 4 hours, a solution of 46.1 g (0.15 mol) of 3-[3-(4-fluoro-phenyl)-1-isopropyl-1H-indol-2-yl]-propenal in 350 ml of tetrahydrofuran is added dropwise (3-[3-(4-fluoro-phenyl)-1-isopropyl-1H-indol-2-yl]-propenal is known and can be prepared, for example, as described in U.S. Pat. No. 5,118,853, Example 6). The cooling bath is removed and there are added, in succession, 15 ml of ethanol, dissolved in 35 ml of tetrahydrofuran, and 100 ml of saturated aqueous ammonium chloride solution. The organic phase is separated off and the aqueous phase is extracted twice using 100 ml of tert-butyl methyl ether each time. The combined organic phases are washed with 200 ml of saturated sodium chloride solution, dried over sodium sulfate and filtered, and the solvent is removed in vacuo.

Yield: 63 g of red, viscous oil (quantitative conversion)

¹H NMR (CDCl₃, 300 MHz): 7.55-7.49 (m, 2H, aryl-CH); 7.42-7.35 (m, 2H, aryl-CH); 7.22-7.04 (m, 4H, aryl-CH); 6.73 (dd, J=16/2 Hz, 1H, olefin-CH); 5.71 (dd, J=16/5 Hz, 1H, olefin-CH); 4.85 (sept, J=7 Hz, 1H, CH(CH₃)₂); 4.62 (br, 1H, CH—OH); 4.18 (q, J=7 Hz, 2H, CH₂O); 3.16 (d, J=4 Hz, 1H, OH); 2.56-2.38 (m, 2H, CH₂); 1.68 (d, J=7 Hz, 6H, CH(CH₃)₂); 1.29 (t, J=7 Hz, 3H, CH₂CH₃).

### EXAMPLE 2

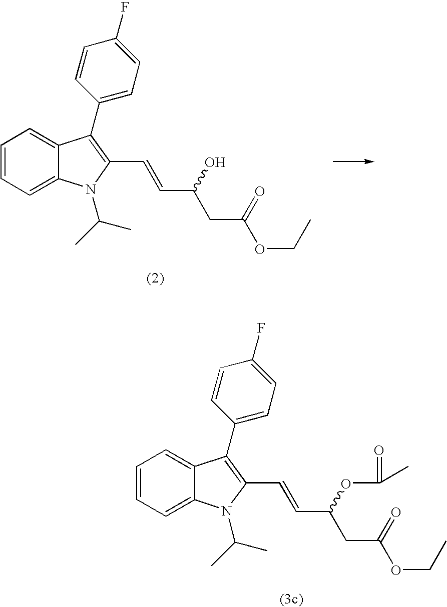

To a solution of 2.11 g of a compound of formula (2), obtainable analogously to Example 1, in 20 ml of acetic anhydride there are slowly added dropwise, at −45° C., 60 μl of scandium(III) trifluoromethanesulfonate solution, Sc(OTf)₃ solution (0.1M in acetonitrile). The cooling bath is removed and the reaction mixture is stirred at room temperature for 24 hours, a further 100 μl of Sc(OTf)₃ solution (0.1M in acetonitrile) being added after 8 hours. The reaction mixture is then extracted in a two-phase mixture of saturated sodium hydrogen carbonate solution and methylene chloride. The organic phase is separated off, washed with saturated sodium chloride solution, dried over magnesium sulfate and concentrated. The crude product is purified by column chromatography on silica gel (petroleum ether/ethyl acetate=2:1), whereupon 2.1 g (in a yield of 91%, based on the amount of compound of formula (2) used) of the compound of formula (3c) are obtained.

HPLC: Chiralcel AD (0.46×25 cm), hexane:EtOH 98:2, 1 ml/min, t_{R}=7.16/8.34 min.

¹H NMR (300 MHz, CDCl₃): 1.27 (3H, t, J=7.3 Hz); 1.68 (6H, 2 d, J=7.0, 7.0 Hz); 2.06 (3H), s);2.53 (1H, dd, J5.9, 15.8 Hz); 2.67 (1H, dd, J=7.3, 15.8 Hz); 4.16 (1H, q, J=7.3 Hz); 4.82 (1H, sept., J=7.0 Hz); 5.62 (1H, dd, J=7.3, 15.5 Hz); 5.71 (1H, m); 6.55 (1H, d, J=15.5 Hz); 7.06-7.15 (3H, m); 7.17-7.24 (1H, m); 7.34-7.41 (2H, m); 7.50-7.56 (2H, m).

¹³C NMR (75 MHz, CDCl₃): 14.2, 21.9, 21.9, 39.3, 47.8, 71.0, 111.4, 115.0, 115.3, 119.5, 119.6, 122.0, 122.5, 131.1 (d, J=3.2 Hz), 131.7, 131.8, 132.5, 132.7, 135.2, 161.3 (d, J=244 Hz), 169.2, 169.5.

### EXAMPLE 3

### 5-[3-(4-Fluorophenyl)-1-isopropyl-1[H]-indol-2-yl]-3-(2-methoxy-acetoxy)-pent-4-enoic acid ethyl ester

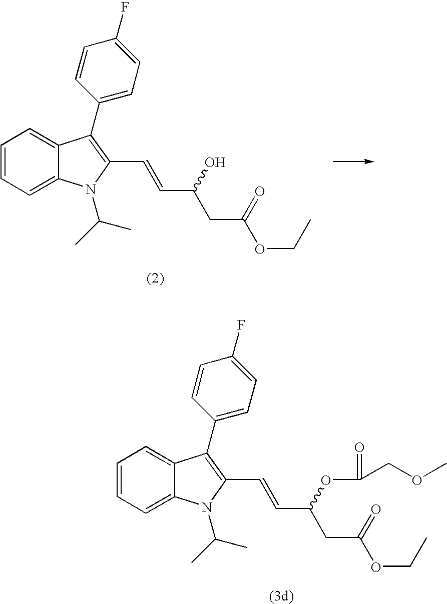

To a solution of 1.0 g of a compound of formula (2), obtainable analogously to Example 1, in 3 ml of acetonitrile there are added, at 0° C., 200 μl of Sc(OTf)₃ (0.1M in acetonitrile) and 5 ml of methoxyacetic anhydride. The cooling bath is then removed and the reaction mixture is stirred at RT for 24 hours. The reaction mixture is diluted with methylene chloride and extracted with saturated sodium chloride solution. The organic phase is dried over sodium sulfate, filtered and concentrated by evaporation. After purification by column chromatography, there are obtained 1.02 g (in a yield of 94%, based on the amount of compound of formula (2) used) of the compound of formula (3d).

HPLC: Chiralcel OD (0.46×25 cm), hexane:EtOH 98:2, 1 ml/min, t_{R}=10.23/11.15 min.

¹H NMR (300 MHz, CDCl₃): 1.27 (3H, t, J=7.3 Hz); 1.67 (6H, dd, J=7.0 Hz); 2.56 (1H, dd, J=5.9, 15.8 Hz); 2.68 (1H, dd, J=7.3, 15.8 Hz); 3.45 (3H, s); 4.00 (2H, dd, J=16.1, 19.0 Hz); 4.15 (2H, q, J=7.3 Hz); 4.81 (1H, sept., J=7.0 Hz); 5.61 (1H, dd, J=7.3, 15.5 Hz); 5.81 (1H, m); 6.78 (1H, d, J=15.5 Hz); 7.05-7.15 (3H, m); 7.17-7.23 (1H, m); 7.33-7.40 (2H, m); 7.49-7.55 (2H, m). ¹³C NMR (75 MHz, CDCl₃): 14.2, 21.9, 39.2, 47.9, 59.3, 60.9, 69.8, 71.5, 111.4, 115.0, 115.3, 119.5, 119.7, 122.0, 123.2, 131.2 (d, J=3.4 Hz), 131.7, 131.8, 132.0, 132.3, 135.2, 159.8 (d, J=244.7 Hz), 168.9, 169.1.

### EXAMPLE 4

### 3-Butyryloxy-5-[3-(4-fluorophenyl)-1-isopropyl-1[H]-indol-2-yl]-pent-4-enoic ethyl ester

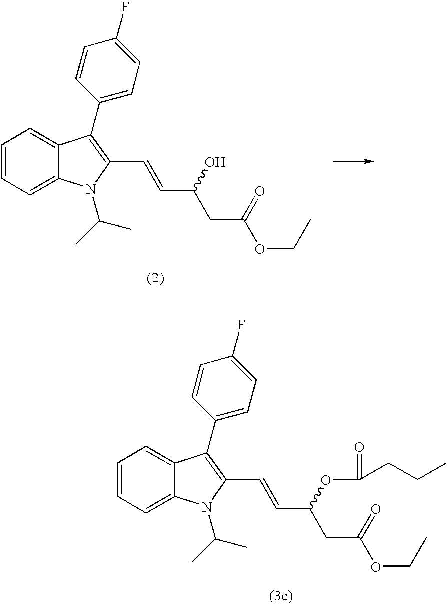

6.0 g (15.0 mmol) of a compound of formula (2), obtainable analogously to Example 1, are dissolved, at 0° C., in 60 ml of dry acetonitrile, and 3.6 g (23 mmol) of butyric anhydride are slowly added. 1.6 ml of dry pyridine are added dropwise, at 0° C., to the solution and the temperature is allowed to rise to room temperature overnight. The mixture is then diluted with 300 ml of diethyl ether and washed in succession with 2N sodium hydroxide solution (3×100 ml), 1N hydrochloric acid solution (3×100 ml), saturated sodium hydrogen carbonate (3×100 ml) and saturated sodium chloride solution. The organic phase is dried over magnesium sulfate and concentrated by evaporation. After purification by column chromatography (petroleum ether:ethyl acetate=2:1) there are obtained 6.55 g (in a yield of 94%, based on the amount of compound of formula (2) used) of the compound of formula (3e).

HPLC: Chiralcel OD (0.46×25 cm), hexane:EtOH 98:2, 1 ml/min, t_{R}=6.0/6.6 min. ¹H NMR (300 MHz, CDCl₃): 1.00 (3H, t, J=7.3 Hz); 1.29 (3H, t, J=7.0 Hz); 1.63-1.76 (8H, m); 2.32 (2H, 2×t, J=7.6 Hz); 2.56 (1H, dd, J=5.9, 15.5 Hz); 2.69 (1H, dd, J=7.6, 15.5 Hz); 4.16 (2H, q, J=7.0 Hz); 4.85 (1H, sept, J=7.0 Hz); 5.66 (1H, dd, J=7.4, 15.9 Hz); 5.74-5.82 (1H, m); 6.76 (1H, dd, J=0.6, 15.9 Hz); 7.07-7.17 (3H, m); 7.19-7.26 (1H, m); 7.38-7.44 (2H), m); 7.52-7.59 (2H, m). ¹³C NMR (75 MHz, CDCl₃): 13.6, 14.8, 18.5, 21.8, 21.9, 36.2, 39.3, 47.8, 60.7, 70.6, 111.4, 114.9, 115.2, 119.4, 119.6, 121.9, 122.4, 131.1 (d, J=7.8 Hz), 131.7, 131.8, 132.8, 159.8 (d, J=244.7 Hz), 169.2, 172.6.

### EXAMPLE 5

### (S)-5-[3-(4-Fluorophenyl)-1-isopropyl-1[H]-indol-2-yl]-3-hydroxy-pent-4-enoic acid ethyl ester

### (S)-1(3a) and (R)-3-acetoxy-5-[3-(4-fluorophenyl)-1-isopropyl-1[H]-indol-2-yl]-pent-4-enoic acid ethyl ester (R)-(3a′)

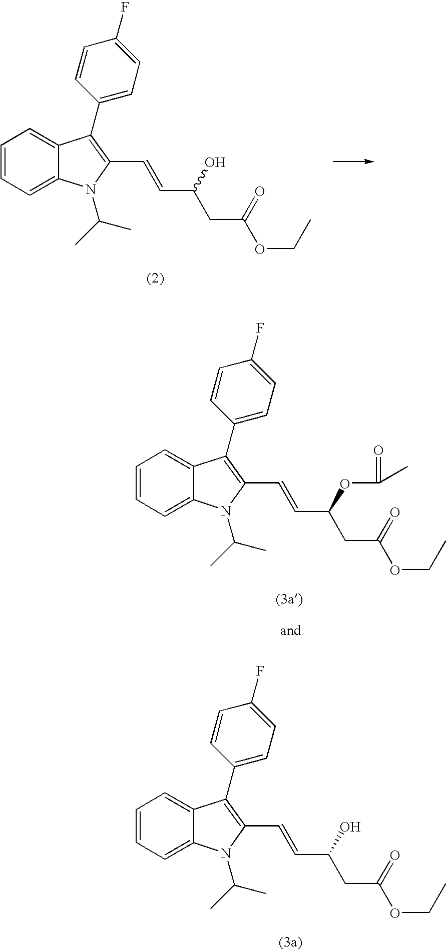

To a solution of 41.0 g of a compound of formula (2), obtainable analogously to Example 1, in 600 ml of tert-butyl methyl ether there are added 29.4 ml of vinyl acetate and 15.0 g of Novozyme 435 lipase and the mixture is shaken at RT for 60 hours. It is then washed three times with saturated sodium hydrogen carbonate solution and once with saturated sodium chloride solution, and the organic phase is dried over sodium sulfate, filtered and concentrated by evaporation. After purification by column chromatography on silica gel (petroleum ether:ethyl acetate=2:1), the two products (3a) and (3a′) are obtained in the form of colourless oils.

16.8 g (in a yield of 46%, based on the compound of formula (2)) of the compound of formula (3a), [α_{D}]=+2.40 (c=5.4, MeOH). HPLC: Chiralcel AD (0.46×25 cm), hexane:EtOH 98:2, 1 ml/min, tR=15.7 min, 97% ee. ¹H NMR (300 MHz, CDCl₃): 1.15 (3H, t, J=7.3 Hz); 1.55 (6H, d, J=7.0 Hz); 2.28-2.44 (2H, m); 3.23 (1H, d, J=4.4 Hz, OH); 4.00 (2H, q, J=7.3 Hz); 4.74 (1H, sept., J=7.0 Hz); 5.62 (1H, dd, J=7.3, 15.5 Hz); 6.63 (1H, d, J=15.5 Hz); 6.92-7.02 (3H, m), 7.03-7.09 (1H, m); 7.24-7.31 (2H, m); 7.38-7.43 (2H, m).

¹³C NMR (75 MHz, CDCl₃): 14.2, 21.7, 41.0, 47.7, 60.7, 68.5, 111.4, 114.9, 115.2, 119.1, 119.2, 119.5, 121.6, 131.6 (d, J=3.4 Hz), 131.6, 131.7, 133.1, 134.9, 137.0, 159.7 (d, J=244.4 Hz), 170.7, 171.7.

20.7 g (in a yield of 47%, based on the amount of compound of formula (2) used) of the compound of formula (3a′), according to NMR analysis. [α_{D}]=+51.80 (c=5, MeOH). HPLC: Chiralcel AD (0.46×25 cm), hexane:EtOH 98:2, 1 ml/min, tR=7.16 min, ≧98% ee. ¹H NMR: identical to the data for the compound of formula (3c).

### EXAMPLE 6

### (S)-5-[3-(4-Fluorophenyl)-1-isopropyl-1[H]-indol-2-yl]-3-hydroxy-pent-4-enoic acid ester

### (S)-1(3a) and (R)-3-butyryloxy-5-[3-(4-fluorophenyl)-1-isopropyl-1[H]-indol-2-yl]-pent-4-enoic acid ethyl ester (R)-(3d)

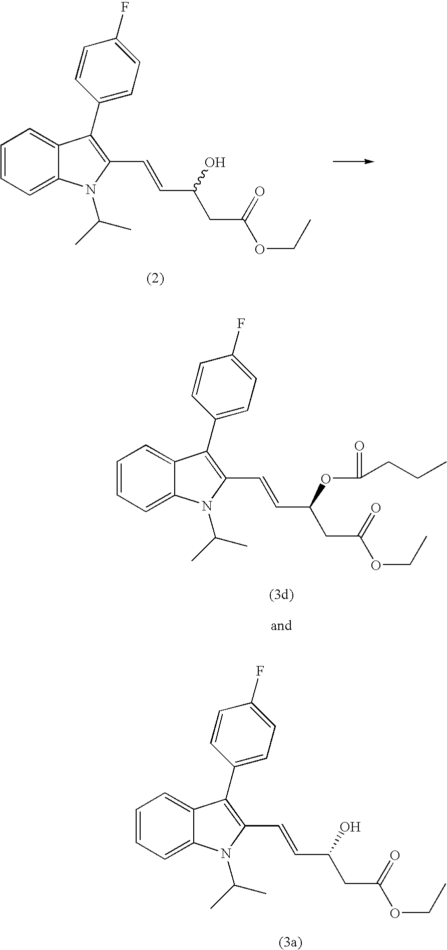

To a solution of 10.2 g of a compound of formula (2), obtainable analogously to Example 1, in 150 ml of tert-butyl methyl ether there are added 8.65 ml of vinyl butyrate and 3.8 g of Novozyme 435 lipase and the mixture is shaken at room temperature for 30 hours. It is then washed three times with saturated sodium hydrogen carbonate solution and once with saturated sodium chloride solution, and the organic phase is dried over sodium sulfate, filtered and concentrated. After purification by column chromatography on silica gel (petroleum ether/ethyl acetate=2:1) the two products are obtained.

4.58 g (in a yield of 44.7%, based on the amount of compound of formula (2) used) of the compound of formula (3a), ¹H NMR: identical to the data for the compound of formula (2). [α_{D}]=+3.10 (c=5.7, MeOH). Chiralcel AD (0.46×25 cm), hexane:EtOH 98:2, 1 ml/min, tR=15.7 min, 97% ee.

5.30 g (in a yield of 44.5%, based on the amount of compound of formula (2) used) of the compound of formula (3d), [α_{D}]=+66.5 (c=2, MeOH). HPLC: Chiralcel OD (0.46×25 cm), hexane:EtOH 98:2, 1 ml/min, tR=6.6 min, ≧98% ee. ¹H NMR (300 MHz, CDCl₃): 0.88 (3H, t, J=7.6 Hz); 1.28 (3H, t, J=7.0 Hz); 1.61-1.71 (8H, m); 2.29 (2H, 2 t, J=7.6 Hz); 2.54 (1H, dd, J=5.8, 15.5 Hz); 2.66 (1H, dd, J=7.6, 15.5 Hz); 4.14 (2H, q, J=7.0 Hz); 4.82 (1H, sept., J=7.0 Hz); 5.64 (1H, dd, J=7.0, 15.8 Hz); 5.74 (1H, m); 6.73 (1H, d, J=15.8 Hz); 7.06-7.15 (3H, m); 7.17-7.23 (1H, m); 7.35-7.42 (2H, m); 7.50-7.56 (2H, m). ¹³C NMR (75 MHz, CDCl₃): 12.7, 13.2, 17.5, 20.8, 35.3, 38.3, 46.8, 59.8, 69.7, 110.4, 114.0, 114.3, 118.4, 118.6, 120.9, 121.4, 126.9, 130.1 (d, J=3.2 Hz), 130.7, 130.8, 131.5, 131.8, 134.1, 160.2 (d, J=243.9 Hz), 168.3, 171.2.

### EXAMPLE 7

### (S)-7-[3-(4-Fluorophenyl)-1-isopropyl-1[H]-indol-2-yl]-5-hydroxy-3-oxo-hept-6-enoic acid tert-butyl ester (S)-3

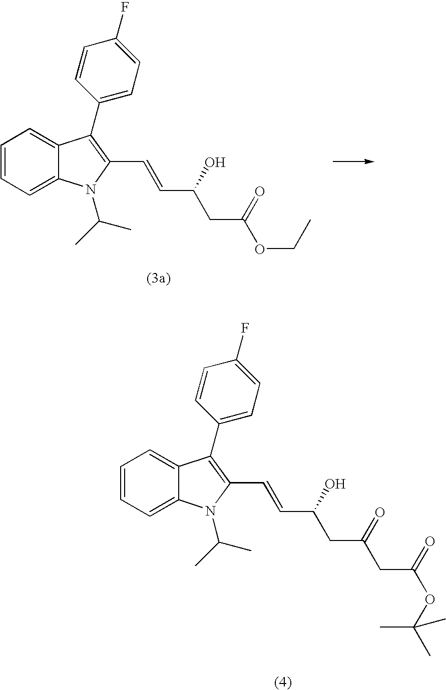

To a solution of 1.2 ml of diisopropylamine in 5 ml of tetrahydrofuran there are added dropwise, at 0° C., 3.1 ml of n-butyllithium (2.7M in heptane). The reaction mixture is stirred at 0° C. for 1 hour and then cooled to −45° C. At that temperature, 1.1 ml (8.0 mmol) of tert-butyl acetate are added dropwise and the reaction mixture is stirred at −40° C. for 1 hour. A solution of the compound of formula (3a) in 3 ml of THF is then added dropwise. The mixture is stirred first at −40° C. for 4 hours and then at 0° C. for 2 hours. 0.5M sodium chloride solution is then added to the reaction mixture, which is then extracted with ether. The organic phase is washed in succession with 1M hydrochloric acid solution, saturated sodium hydrogen carbonate solution and sodium chloride solution, dried over magnesium sulfate and concentrated by evaporation. The crude product is then purified by column chromatography on silica gel (petroleum ether/ethyl acetate=2:1), whereupon 797 mg (in a yield of 86%, based on the amount of compound (3a) used) of the compound of formula (4) are obtained in the form of a yellow syrup. According to NMR analysis the compound of formula (4) is pure.

HPLC: Chiralcel OD (0.46×25 cm), hexane:EtOH 98:2, 1 ml/min, tR=24.5 min, 94% ee. [α_{D}]=−1.82 (c=0.5, MeOH). ¹H NMR (300 MHz, CDCl₃): 1.51 (9H, s); 1.69 (6H, d, J=7.0 Hz); 2.62-2.78 (2H, m); 3.34 (1H, OH); 3.40 (2H, s); 3.38-4.76 (1H, m); 4.89 (1H, sept., J=7.0 Hz); 5.76 (1H, dd, J=5.3, 15.8 Hz); 6.75 (1H, dd, J=1.7, 15.8 Hz); 7.06-7.17 (3H, m); 7.18-7.24 (1H, m); 7.39-7.46 (2H, m); 7.53-7.78 (2H, m). ¹³C NMR (75 MHz, CDCl₃); 22.1, 28.3, 48.1, 49.2, 51.5, 68.4, 82.7, 119.9, 115.3, 115.6, 119.4, 119.6, 119.9, 122.0, 128.6, 132.2 (d, J=3.2 Hz), 133.6, 135.3, 137.4, 161.2 8d, J=243.9 Hz), 166.2, 171.3, 203.3.

### EXAMPLE 8

### (3R, 5S)-7-[3-(4-Fluorophenyl)-1-isopropyl-1[H]-indol-2-yl]-3,5-dihydroxy-hept-6-enoic acid tert-butyl ester (3R,5S)-5

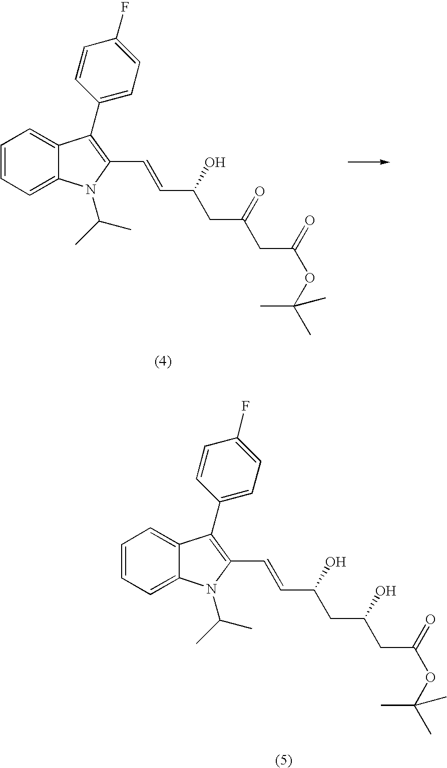

To a solution of 8 ml of tetrahydrofuran and 2.5 ml of methanol there are added, at room temperature and under argon, 1.75 ml of triethyl borate (1M in tetrahydrofuran) and the mixture is stirred at room temperature for 4 hours. The mixture is then cooled to −78° C. and a solution of 725 mg (1.56 mmol) of the compound of formula (4) in 3 ml of tetrahydrofuran is added dropwise. 65 mg (1.72 mmol) of sodium borohydride are then added thereto in a single portion. Stirring is then carried out at −78° C. for one hour, 2.5 ml of 1M ammonium chloride are then added and the reaction mixture is extracted with ethyl acetate. The organic phase is dried over magnesium sulfate and concentrated by evaporation. The yellow residue is dissolved at 0° C. in 5 ml of tetrahydrofuran and slowly oxidised with 1 ml of hydrogen peroxide (33%) until borate ester can no longer be detected in a thin-layer chromatogram. The reaction mixture is then diluted with ethyl acetate and extracted with saturated sodium chloride solution. The organic phase is dried over magnesium sulfate and concentrated by evaporation, the residue being taken up several times in methanol and then concentrated by evaporation in a rotary evaporator at 40-50° C. The compound of formula (5) is obtained in that manner in the form of a light-yellow foam. According to NMR the compound of formula (5) is pure.

HPLC: Chiralcel AD (0.46×25 cm), hexane:EtOH 98:2, 1 ml/min, tR=28.9 min. (syn:anti=98.8:1.2, syn: 97% ee). [α_{D}]=+56.0 (c=0.4, MeOH). ¹H NMR (300 MHz, CD₃OD): 1.35-1.42 (11H, m); 1.57 (6H, dd, J=7.0, 7.0 Hz); 3.89-4.00 (1H, m); 4.30 (1H, dq, J=11.0, 6.8 Hz); 4.86 (1H, sept. J=7.0 Hz); 5.66 (1H, dd, J=6.5, 16.1 Hz); 6.60 (1H, dd, J=1.1, 16.1 Hz); 6.89-6.95 (1H, m); 7.00-7.08 (3H, m); 7.31-7.37 (3H, m); 7.45-7.49 (1H, m). ¹³C NMR (75 MHz, CD₃OD): 21.9, 22.0, 44.4, 67.2, 71.3, 81.7, 112.6, 115.3, 115.8, 116.1, 119.9, 120.1, 120.4, 122.5, 129.4 8d, J=3.2 Hz), 132.9, 132.9, 134.6, 136.2, 140.3, 161.1 8d, J=243.9 Hz), 172.3.

The product can be recrystallised from methylene chloride/hexane, whereupon colourless crystals are obtained which according to HPLC have the following purity: 100% de, 100% ee.

1. A process for the preparation of a compound of formula (1) 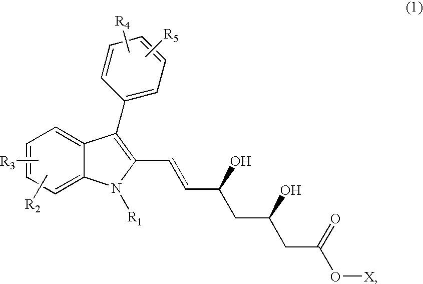
wherein R₁ is unsubstituted or substituted C₁-C₈alkyl,
R₂, R₃, R₄ and R₅ are each independently of the others hydrogen, unsubstituted or substituted C₁-C₈alkyl, C₁-C₈alkoxy, phenoxy or benzyloxy, or halogen, and
X is hydrogen, an organic radical or a cation,
which process comprises reacting a compound of formula (2) 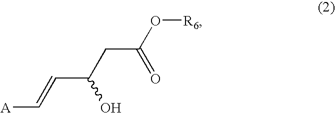
wherein R₆ is an organic radical and
A is an organic radical of formula (1a) 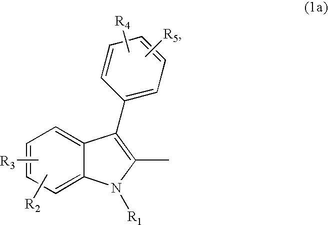
wherein R₁, R₂, R₃, R₄ and R₅ are as defined hereinbefore,
by enzymatic acylation to form compounds of formulae (3a) and (3a′) 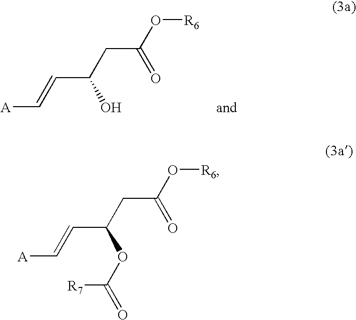
wherein
A is an organic radical of formula (1a) and
R₆ and R₇ are each independently of the other an organic radical, and
then reacting the compound of formula (3a) with a compound introducing the radical of formula CH₂—COOR₈, R₈ being an organic radical, and
then reducing, and optionally hydrolysing, the resulting compound of formula (4)

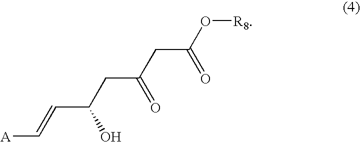

2. A process according to claim 1, wherein
X is hydrogen, C₁-C₈alkyl which is unsubstituted or substituted by phenyl, or a cation.

3. A process according to claim 1, wherein
X is a sodium cation.

4. A process for the preparation of compounds of formulae (3a) and (3a′) as defined in claim 1, which process comprises reacting a compound of formula (2) 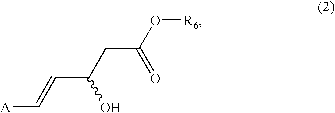
wherein R₆ is an organic radical and
A is an organic radical of formula (1a) 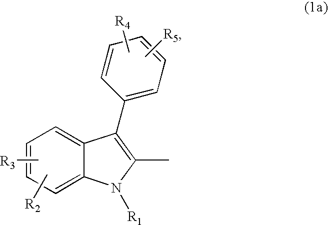
wherein R₁ is unsubstituted or substituted C₁-C₈alkyl,
R₂, R₃, R₄ and R₅ are each independently of the others hydrogen, unsubstituted or substituted C₁-C₈alkyl, C₁-C₈alkoxy, phenoxy or benzyloxy, or halogen,
by enzymatic acylation to form compounds of formulae (3a) and (3a′) 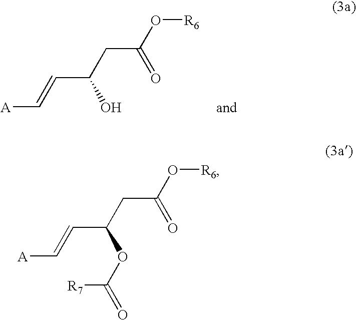
wherein
A is an organic radical of formula (1a) and
R₆ and R₇ are each independently of the other an organic radical.

5. A process according to claim 1, wherein R₁ is isopropyl.

6. A process according to claim 1, wherein R₂, R₃ and R₅ are hydrogen and R₄ is fluorine bonded in the 4-position.

7. A process according to claim 1, wherein the enzymatic acylation is carried out in the presence of a lipase.

8. A process according to claim 1, wherein the enzymatic acylation is carried out in the presence of the enzyme Novozyme 435 or Lipozyme RM-IM.

9. A process according to claim 1, wherein the enzymatic acylation is carried out in the presence of immobilised or insoluble enzymes.

10. A process according to claim 1, wherein the enzymatic acylation is carried out in the presence of immobilised or insoluble enzymes in a continuous process.

11. A process according to claim 1, wherein R₆ is an unsubstituted or substituted C₁-C₆alkyl radical.

12. A process according to claim 1, wherein R₆ is ethyl.

13. A process according to claim 1, wherein R₇ is an unsubstituted or substituted C₁-C₆alkyl radical.

14. A process according to claim 1, wherein R₇ is methyl, ethyl, n-propyl, methoxyethyl or methoxymethyl.

15. A compound of formula (3a) 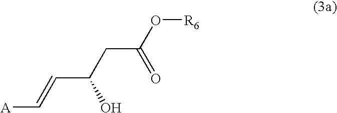
wherein
R₆ is ethyl and
A is an organic radical of formula (1a) 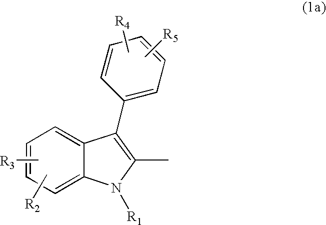
wherein R₁ is unsubstituted or substituted C₁-C₈alkyl,
R₂, R₃, R₄ and R₅ are each independently of the others hydrogen, unsubstituted or substituted C₁-C₈alkyl, C₁-C₈alkoxy, phenoxy or benzyloxy, or halogen.

16. A compound of formula (3a′) 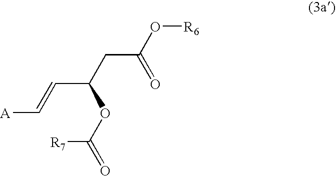
wherein
R₆ and R₇ are each independently of the other an organic radical and
A is an organic radical of formula (1a) 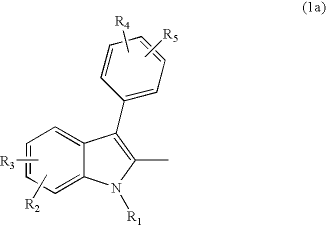
wherein R₁ is unsubstituted or substituted C₁-C₈alkyl,
R₂, R₃, R₄ and R₅ are each independently of the others hydrogen, unsubstituted or substituted C₁-C₈alkyl, C₁-C₈alkoxy, phenoxy or benzyloxy, or halogen.

17. A compound of formula (3a′) according to claim 16, wherein R₇ is methyl, ethyl, n-propyl, a methyl ethyl ether radical, a methyl methyl ether radical or an ethyl methyl ether radical.

18. A compound of formula (3a′) according to claim 16, wherein R₆ is ethyl or isopropyl.

19. (canceled)

20. A process according to claim 4, wherein the enzymatic acylation is carried out in the presence of immobilised or insoluble enzymes in a continuous process.

21. A process according to claim 4, wherein R₆ is ethyl and wherein R₇ is methyl, ethyl, n-propyl, methoxyethyl or methoxymethyl.
